(19) United States
(12) Patent Application Publication
(10) Pub. No.: US 2004/0138600 A1
(43) Pub. Date: Jul. 15, 2004

(54) Pads particularly back-support bandages

(76) Inventor: Thorsten Herzberg, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/470,375

(22) PCT Filed: Jan. 24, 2002
(86) PCT No.: PCT/EP02/00707

(30) Foreign Application Priority Data
Jan. 26, 2001 (DE) 101 03 545.4

(51) Int. Cl. A61F 13/00

(52) U.S. Cl. 602/41

(57) ABSTRACT

Pad, in particular for a back bandage with an anterior side facing the body and a posterior side facing away from the body, with the anterior side of the pad being provided with at least one dam-shaped, linear elevation, which is oriented substantially parallel to the sagittal axis, and with both ends of the dam-shaped elevation comprising at least one raised friction point.

The invention relates to pads, in particular for back support bandages.

Depending on their construction and their field of indication, orthopedic bandages exercise a fixing, guiding, propping, and/or supporting function on the extremities of the human body.

These medical bandages must have a shape to conform to the anatomical conditions for being able to externally act upon the human body in a formfitting and frictional manner.

Medical bandages of this kind are produced by preparing cuts of flat materials, for example, of neoprene, knits, or woven fabrics. The anatomy-conforming shape is realized by the form of the cuts or by darts, for example, gussets, and by the subsequent assembly of the cuts as is also common in the case of apparel.

The assembly may also occur by sewing, gluing, or other common methods. The great disadvantage of these bandages is that the exact anatomical fit can be achieved only with great difficulties, and a plurality of joints results, for example, seams. These joints change the properties of the material in use, and there is a risk or pressure sores on the skin.

The generally known pads for back support bandages have a rigid shape or a certain softness by a silicone rubber coating.

Because of the rigidity of the pads, this results in the case of the former in a reduction of the wearing comfort and an increased tendency to pressure and chafing marks by the hard edges.

The silicone rubber pads exhibit an improved wearing comfort, but tend to slide in the caudal direction because of their high dead weight. In individual cases, the bandage tends to migrate caudally together with the pad. A further problem lies in that increased perspiration is found on the patient in the region of the pad.

DE 36 13 235 A1 discloses a back support bandage with a back portion and lateral abdominal bands. The back portion consists, for example, of neoprene, the abdominal bands of a longitudinally elastic material. As an option, the bandage includes abdominal lifting bands, which are made in the same way as the above-described draw bands, and likewise as an option, it includes a back pad.

As can be noted from U.S. Pat. No. 5,429,587, EP 0 619 103 A1, or DE 27 22 563 A1, there exist specially shaped pads, which are used in back bandages.

DE 25 06 647 C1 discloses a back support bandage, with an elastic band that continuously extends over the back region and the side regions, and which is provided with support elements in the region of the spinal column.

It is an object of the invention to devise a semirigid, functionally molded pad, which has a great wearing comfort with a low dead weight, but is simultaneously simple and uncomplicated to apply by the patient, and which has a high degree of dynamics. Furthermore, the pad is to excel in a back bandage, which in turn is to be produced in a cost-favorable manner.

This object is accomplished by a bandage characterized in claim 1. The subject matter of the dependent claims concerns advantageous further developments of the pad, as well as a back bandage provided with the pad.

Accordingly, the invention describes a pad, in particular for a back bandage, with an anterior side facing the body, and a posterior side facing away from the body, with the anterior side of the pad being provided with at least one dam-shaped linear elevation, which is oriented substantially parallel to the sagittal axis. The two ends of the dam-shaped elevation comprise at least one raised friction point.

The friction point may have a pointed or circular configuration. In combination with the preferred configuration of the elevations toward the side with different heights of the contour, this will lead to the preferably desired friction of the medial and lateral strands of the back muscles.

In a further advantageous development of the pad, the dam-shaped elevation is undulated.

In a further advantageous development of the pad, the dam-shaped elevation extends in mirror-symmetric relationship with the sagittal axis.

In a further advantageous development of the pad, same comprises three dam-shaped elevations, one extending below the other.

To make pads according to the invention, different materials may be used, such as, for example, spacer fabrics, foams, nonwovens, and comparable materials.

Spacer fabrics are disclosed in EP 0 071 212 B1. Spacer fabrics are matted layered structures comprising a cover layer of a fiber or filament nonwoven, a base layer, and between these layers, individual spacer fibers or tufts thereof, which are needle punched through the particle layer in distributed relationship over the surface of the layered structure, and which interconnect the cover layer and the base layer. As an additional, but not required characteristic, the spacer fibers according to EP 0 071 212 B1 include particles of inert mineral particles, such as, for example, sand, gravel, or the like.

The spacer fibers that are needle punched through the particle layer keep the cover layer and the base layer in spaced relationship, and they connect to the cover layer and the base layer.

Woven or knit spacer fabrics are described, among other things, in two articles, namely in an article entitled “Raschelgewirkte Abstandsgewirke” (Raschel-Knit Spacer Fabrics) that was published in the trade journal “kettenwirk-praxis 3/93, 1993, pp. 59-63, and an article entitled “Raschelgewirkte Abstandsgewirke” (Raschel-Knit Spacer Fabrics) that was published in the trade journal “kettenwirk-praxis 1/94”, 1994, pages 73-76. The contents of these articles are herewith incorporated by reference and become part of this disclosure and invention.

Furthermore, for making pads, it is possible to use in accordance with the invention materials of different strengths, volume weights, etc.

In another advantageous further development of the pad, the pad is surrounded on one side, on both sides, or completely by a textile fabric for increasing the wearing comfort.

In another advantageous further development of the pad, the pad is perforated, for example, by a plurality holes that are arranged in a pattern and have a diameter from 1 to 5 mm, for increasing the breathing activity of the pad.

Pads of the invention may be made by different methods.

When the pads are of foam rubber, they may be pressed by the compression molding method to different shapes and thickness. This process is intended to modify the elastic properties of the material locally by the different density of the foam material after the deformation. To this end, reference may be made to WO 95/32690 A1, which describes the method in great detail.

Furthermore, it is possible to mold thermoplastic plastic sheet material in an anatomy-conforming way to the pads of the invention. These materials, such as, for example, polyethylene (HDPE), polypropylene (PP), or a copolymer possess a thermoplastic transition range from about 170° to 250° C.

In a particularly advantageous manner, the pads, whose raw material comprises at least 10 wt. % thermoplastic material, may be made by a three-dimensional heat fitting process.

In this process, the raw material is preferably a thermoplastically deformable spacer knit, a thermoplastically deformable nonwoven, a thermoplastically deformable foam material, and/or a thermoplastically deformable plastic of a low rigidity. Possible plastics, which have such a low rigidity, are polyethylene (LDPE) and polypropylene.

Preferably, the raw material is also a thermoplastically deformable fabric or knit, wherein elastic fibers or components may be integrated. The described materials may thus be elastic by incorporating, for example elastane or elastodiene.

It is further preferred to combine the raw material to two- or multilayer laminates, with the lamination being carried out by means of generally known methods, for example, bonding.

Advantageously, the pad of the invention may be produced by heating the raw material that has been cut to a corresponding fit, to the thermoplastic softening range, and by subsequently compressing the raw material by means of a correspondingly shaped positive mold to the required shape.

In a further advantageous manner, the pad is made by heating the raw material that has been cut to a corresponding fit, to the thermoplastic softening range between a positive mold and a negative mold, which are correspondingly shaped, and by compressing it to the required shape. The heating may occur by means of heatable molds, or also in that the raw material is first heated in an oven to a corresponding temperature, and only then shaped in the molds.

It has been found especially advantageous to use a softer foam material toward the body side and a harder variant for the bandage to thus increase the wearing comfort on the side of the skin.

The semirigid construction accomplishes an optimal adaptation of the pad, in particular to the back of the wearer with a simultaneous supporting effect on the covering portion of the spinal column.

The concept of the invention thus encompasses a back support bandage with at least one of the pads according to the invention.

In a first advantageous embodiment of the back support bandage, same comprises a back portion, lateral abdominal bands, and a closure, in particular a hook and loop closure for joining the abdominal bands, with the back portion being formed of a substantially inelastic material, and the abdominal bands of a substantially single stretch, in particular elastic material.

Preferably, the direction of elongation of the material forming the abdominal bands extends from the back portion to the closure ends.

Preferably, the back portion further comprises on its inner side facing the spinal column a hook and loop tape for receiving the exchangeable pads.

Furthermore, on its sides receiving the closure parts, the abdominal bands may comprise abdominal reinforcements, which in turn are made in particular as thin plastic sheets or slats.

In a further, preferred embodiment of the back bandage, the outer side facing away from the spinal column is provided with abdominal lifting bands that extend over the abdominal bands, and which in turn are advantageously formed of a substantially inelastic material.

In a further preferred embodiment of the back bandage, two reinforcement slats are provided, which extend substantially parallel to the spinal column.

The present back bandage enables a high formfitting engagement between the pad being held and the segments of the spinal column on the one hand, a satisfactory fit and a flat support across the abdominal region, and furthermore an excellent stabilization of the lumbar vertebra segments and the sacrum.

The invention is described in greater detail with reference to two Figures showing an embodiment, and without intending to limit the invention needlessly. In the Figures:

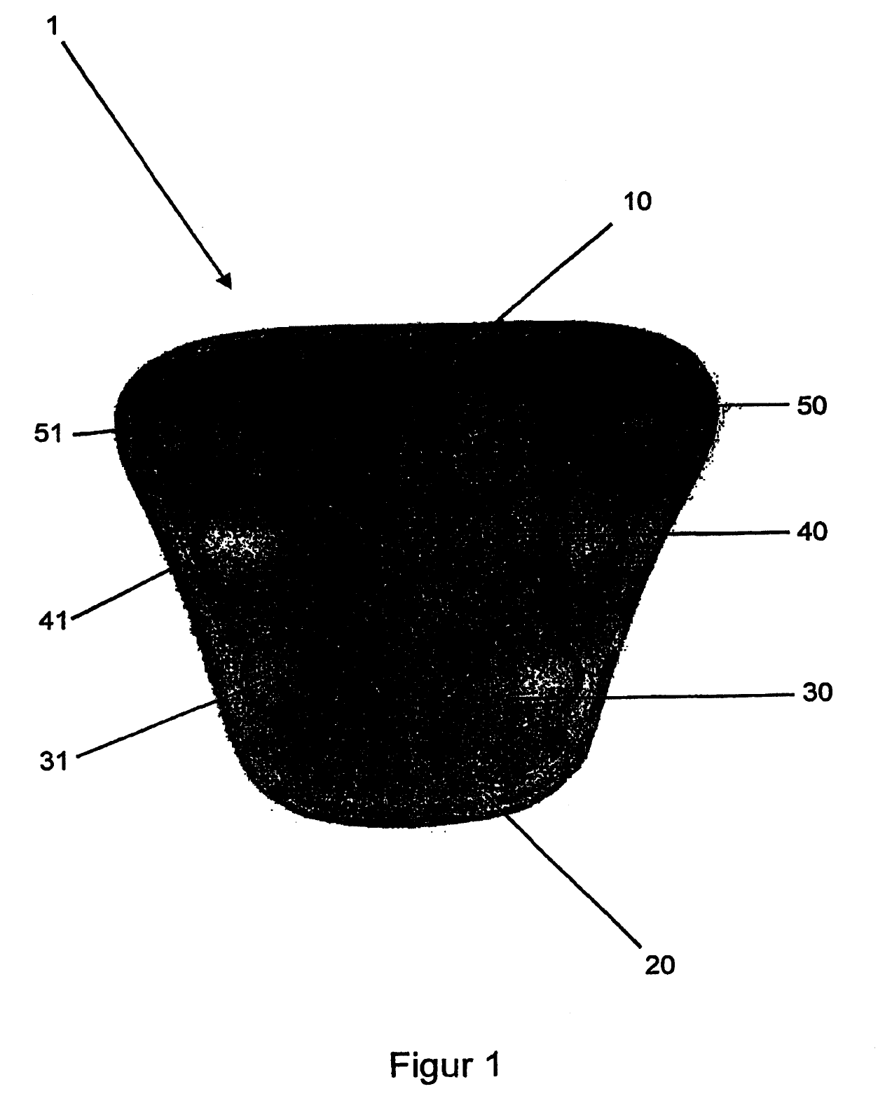

FIG. 1 is an anterior view of the pad; and

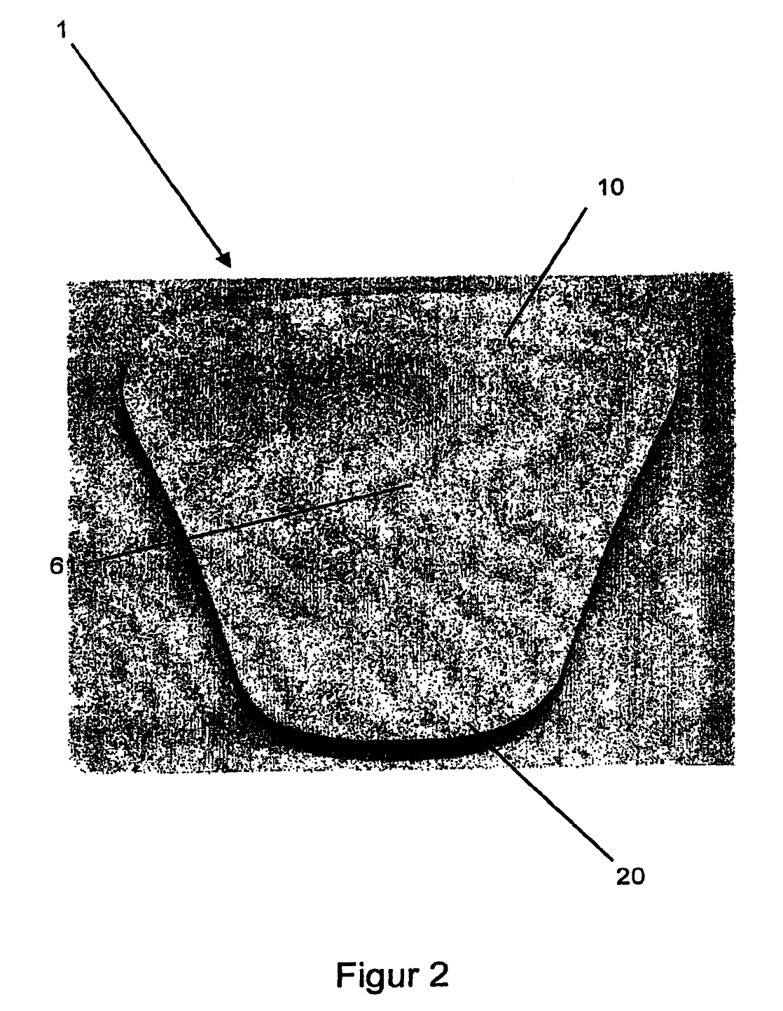

FIG. 2 is a posterior view of the pad.

FIG. 1 is an anterior view of a particularly advantageous embodiment of a pad 1 in particular for a back support bandage. The pad 1 comprises an anterior side facing the body and a posterior side facing away from the body. The anterior side of the pad 1 includes three dam-shaped, linear elevations 30, 40, 50, which are oriented substantially parallel to a horizontal axis. Each of the two ends of the dam-shaped elevations 30, 40, 50 includes a raised friction point 31, 41, 51.

The linear elevations 30, 40, 50 are molded in the shape of a wave. Furthermore, the elevations have an increasing height of their contour toward the sides.

The pad 1 is made substantially trapezoidal, with the pad narrowing from an upper region 10 to a lower region 20.

In the center, the linear elevations 30, 40, 50 have a height from 1.5 mm to 12 mm, preferably 4.5 mm to 8 mm, as well as a width from 10 mm to 30 mm. preferably 12 to 25 mm.

The friction point 31, 41, 51 has a diameter from 10 to 40 mm, preferably 20 mm to 30 mm, as well as a height from 2 mm to 20 mm, preferably 4.5 mm to 10 mm.

FIG. 2 is a posterior view of the pad of figure 1. Arranged in the center of the pad 1 is a hook and loop strip 61, which serves to secure the pad 1 to a back bandage. On its side facing the body, the pad 1 is covered with soft, liquid-absorbing layer of a fabric for increasing the comfort of the patient.

1. Pad, in particular for a back bandage with an anterior side facing the body and a posterior side facing away from the body, with the anterior side of the pad being provided with at least one dam-shaped linear elevation, which is oriented substantially parallel to the sagittal axis, and with both ends of the dam-shaped elevation comprising at least one raised friction point.

2. Pad of claim 1, characterized in that the dam-shaped elevation extends in the shape of a wave.

3. Pad of claims 1 and 2, characterized in that the dam-shaped elevation extends in mirror-symmetric relationship with the sagittal axis.

4. Pad of claims 1-3, characterized in that three dam-shaped elevations are arranged on the pad, one below the other.

5. Pad of at least one of the foregoing claims, characterized in that the raw material for the pad is a thermoplastically deformable spacer knit, a thermoplastically deformable nonwoven, a thermoplastically deformable foam material, and/or a thermoplastically deformable plastic of a low rigidity.

6. Pad of at least one of the foregoing claims, characterized in that the raw material for the pad is a thermoplastically deformable fabric or knit, into which fibers or components may be integrated.

7. Method of making a pad according to at least one of the foregoing claims, characterized in that the raw material is adapted by a three-dimensional heat fitting operation to the anatomy of the predetermined body part.

8. Method of making a pad according to at least one of the foregoing claims, characterized in that the raw material having been cut to the corresponding fit is heated to the thermoplastic softening range, and that the raw material is compressed to the required shape by means of a positive mold that is formed in accordance with the anatomical conditions of the particular body part.

9. Method of making a pad according to at least one of the foregoing claims, characterized in that the raw material having been cut to the corresponding fit is heated to the thermoplastic softening range between a positive mold and a negative mold, which are formed in accordance with the anatomical conditions of the particular body part, and compressed to the required shape.

10. Back support bandage with at least one of the pads according to at least one of the foregoing claims.

11. Back support bandage of claim 10, with a back portion and lateral, in particular elastic abdominal bands, with the back portion being formed of a substantially inelastic material and the abdominal bands of a substantially single stretch material.
